(19) United States
(12) Patent Application Publication
(10) Pub. No.: US 2022/0273374 A1
(43) Pub. Date: Sep. 1, 2022

(54) MEDICAL SHEATH AND SYSTEMS AND METHODS FOR USING MEDICAL SHEATH

(71) Applicant: Baylis Medical Company Inc., Montreal (CA)

(72) Inventor: Gareth Davies, Toronto (CA)

(21) Appl. No.: 17/632,610

(22) PCT Filed: Aug. 14, 2020
(86) PCT No.: PCT/IB2020/057685

§ 371 Date: Feb. 3, 2022

Related U.S. Application Data
(60) Provisional application No. 62/886,534, filed on Aug. 14, 2019.

(51) Int. Cl. A61B 34/20; A61B 8/00; A61B 18/14

(52) U.S. Cl. CPC A61B 34/20; A61B 8/445; A61B 18/1492; A61M 25/0108

(57) ABSTRACT

A medical sheath includes an elongate member having a sidewall extending longitudinally between a proximal end portion of the elongate member and an opposed distal end portion of the elongate member, and radially between an outer surface of the elongate member and an inner surface of the elongate member. A lumen is defined by the inner surface and extends through the elongate member from the proximal end portion to the distal end portion. At least a first echogenic band is associated with the distal end portion and is secured to the sidewall.

### FIELD

This document relates to medical sheaths, such as those used in cardiac procedures. More specifically, this document relates to medical sheaths, methods for carrying out medical procedures using medical sheaths, and medical ultrasound systems including medical sheaths.

### SUMMARY

The following summary is intended to introduce the reader to various aspects of the detailed description, but not to define or delimit any invention.

According to some aspects, a medical sheath includes an elongate member having a sidewall extending longitudinally between a proximal end portion of the elongate member and an opposed distal end portion of the elongate member, and radially between an outer surface of the elongate member and an inner surface of the elongate member. A lumen is defined by the inner surface and extends through the elongate member from the proximal end portion to the distal end portion. At least a first echogenic band is associated with the distal end portion and is secured to the sidewall.

In some examples, the first echogenic band includes a first coil of echogenic material extending around at least a first portion of the elongate member.

In some examples, the medical sheath includes a second echogenic band associated with the distal end portion and spaced from the first echogenic band. The second echogenic band can include a second coil of echogenic material extending around at least a second portion of the elongate member.

In some examples, the medical sheath includes a third echogenic band associated with the distal end portion and spaced from the first echogenic band and the second echogenic band. The third echogenic band can include a third coil of echogenic material extending around at least a third portion of the elongate member.

In some examples, the echogenic band includes a braid of echogenic material extending around at least a first portion of the elongate member.

In some examples, the echogenic band is embedded in the sidewall. The elongate member can include an inner liner and an outer tube, and the echogenic band can be positioned between the inner liner and outer tube.

In some examples, the first echogenic band includes a band of echogenic-filled polymer.

According to some aspects, a medical ultrasound system includes a 3-dimensional ultrasound catheter for emitting ultrasound signals to an anatomical volume and receiving reflected ultrasound signals from the anatomical volume. The system further includes a medical sheath having a distal end portion that is at least partially echogenic, for reflecting the ultrasound signals emitted by the 3-dimensional ultrasound catheter. The system further includes an ultrasound data processor for receiving ultrasound data from the 3-dimensional ultrasound catheter, based on the reflected ultrasound signals, and processing the ultrasound data. The system further includes an imaging system connected to the ultrasound data processor for generating a 3-dimensional visual model representing the positioning of the distal end portion of the medical sheath within the anatomical volume, based on the processed data.

In some examples, the medical sheath includes an elongate member having a sidewall extending longitudinally between a proximal end portion of the elongate member and an opposed distal end portion of the elongate member, and radially between an outer surface of the elongate member and an inner surface of the elongate member; a lumen defined by the inner surface and extending through the elongate member from the proximal end portion to the distal end portion; and at least a first echogenic band associated with the distal end portion and secured to the sidewall.

In some examples, the first echogenic band includes a first coil of echogenic material extending around at least a first portion of the elongate member.

In some examples, the medical sheath includes a second echogenic band associated with the distal end portion and spaced from the first echogenic band. The second echogenic band can include a second coil of echogenic material extending around at least a second portion of the elongate member.

In some examples, the medical sheath includes a third echogenic band associated with the distal end portion and spaced from the first echogenic band and the second echogenic band. The third echogenic band can include a third coil of echogenic material extending around at least a third portion of the elongate member.

In some examples, the first echogenic band includes a braid of echogenic material extending around at least a first portion of the elongate member.

In some examples, the first echogenic band is embedded in the sidewall. The elongate member can include an inner liner and an outer tube, and the echogenic band can be positioned between the inner liner and outer tube.

In some examples, the first echogenic band includes a band of echogenic-filled polymer.

According to some aspects, a method for carrying out a cardiac procedure includes: a. inserting a 3-dimensional ultrasound catheter into patient's body and using the 3-dimensional ultrasound catheter to create a visual model of the patient's heart; b. inserting a medical sheath into the patient's heart and advancing a distal end portion of the medical sheath towards a target location in the patient's heart, wherein the distal end portion of the sheath is at least partially echogenic; c. using the 3-dimensional ultrasound catheter to determine a location of the distal end portion of the medical sheath with respect to the patient's heart; and d. adding a representation of the distal end portion to the visual model of the patient's heart.

In some examples, step d. includes generating an en-face view of the patient's heart from the distal end portion.

In some examples, step d. includes generating a side view of the distal end portion and an area of the patient's heart adjacent the distal end portion.

In some examples, step d. includes generating a rear-view of the distal end portion and an area of the patient's heart adjacent the distal end portion.

In some examples, the method further includes e. advancing a treatment device out of the distal end portion towards the target location, and f. using the treatment device to treat the target location.

In some examples, prior to step e., the method includes adding to the visual model a representation of a predicted contact location of the treatment device and the patient's heart.

In some examples, the treatment device is a radiofrequency perforation device and the target location is a fossa ovalis.

In some examples, the treatment device is a balloon-based system and the target location is a pulmonary vein.

According to some aspects, a kit of parts for a carrying out a cardiac procedure includes a medical sheath, and a radiofrequency perforation device. The medical sheath includes: an elongate member having a sidewall extending longitudinally between a proximal end portion of the elongate member and an opposed distal end portion of the elongate member, and radially between an outer surface of the elongate member and an inner surface of the elongate member; a lumen defined by the inner surface and extending through the elongate member from the proximal end portion to the distal end portion; and at least a first echogenic band associated with the distal end portion and secured to the sidewall. The radiofrequency perforation device has a perforating tip, and is receivable in the lumen with the perforating tip at the distal end portion.

In some examples, the first echogenic band includes a first coil of echogenic material extending around at least a first portion of the elongate member.

In some examples, the kit further includes a second echogenic band associated with the distal end portion and spaced from the first echogenic band. The second echogenic band can include a second coil of echogenic material extending around at least a second portion of the elongate member.

In some examples, the kit further includes a third echogenic band associated with the distal end portion and spaced from the first echogenic band and the second echogenic band. The third echogenic band can include a third coil of echogenic material extending around at least a third portion of the elongate member.

In some examples, the echogenic band includes a braid of echogenic material extending around at least a first portion of the elongate member.

In some examples, the echogenic band is embedded in the sidewall. The elongate member can include an inner liner and an outer tube, and the echogenic band can be positioned between the inner liner and outer tube.

In some examples, the first echogenic band includes a band of echogenic-filled polymer.

According to some aspects, an echogenic device includes an elongate member having a solid body extending longitudinally between a proximal end portion of the elongate member and an opposed distal end portion of the elongate member. The echogenic device includes at least a first echogenic band associated with the distal end portion and secured to the solid body of the elongate member.

According to some aspects, a medical ultrasound system includes a 3-dimensional ultrasound catheter for emitting ultrasound signals to an anatomical volume and receiving reflected ultrasound signals from the anatomical volume. The medical ultrasound system further includes an echogenic device having a distal end portion that is at least partially echogenic for reflecting the ultrasound signals emitted by the 3-dimensional ultrasound catheter. The system further includes an ultrasound data processor for receiving ultrasound data from the 3-dimensional ultrasound catheter and processing the ultrasound data, wherein the ultrasound data is based on the reflected ultrasound signals. The system further includes an imaging system connected to the ultrasound data processor for generating a 3-dimensional visual model representing the positioning of the distal end portion of the catheter within the anatomical volume, based on the processed data.

According to some aspects, a method for carrying out a cardiac procedure includes: a. inserting a 3-dimensional ultrasound catheter into patient's body and using the 3-dimensional ultrasound catheter to create a visual model of the patient's heart; b. inserting the echogenic device into the patient's heart and advancing a distal end portion of the echogenic device towards a target location in the patient's heart, wherein the distal end portion of the echogenic device is at least partially echogenic; c. using the 3-dimensional ultrasound catheter to determine a location of the distal end portion of the medical sheath with respect to the patient's heart; and d. adding a representation of the distal end portion to the visual model of the patient's heart.

According to some aspects, a kit of parts for a carrying out a cardiac procedure includes an echogenic device, and a radiofrequency perforation device. The echogenic device includes: an elongate member having a solid body extending longitudinally between a proximal end portion of the elongate member and an opposed distal end portion of the elongate member; and at least a first echogenic band associated with the distal end portion. The radiofrequency perforation devices includes a perforating tip.

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a perspective view of an example medical ultrasound system;

FIG. 2A is a side view of the sheath of FIG. 1;

FIG. 2B is an enlarged cutaway view of the distal end portion of the sheath of FIG. 2A;

FIG. 2C is a cross-section taken through the distal end portion of the sheath of FIG. 2A;

FIG. 3A is an enlarged cutaway view of a distal end portion of another example sheath;

FIG. 3B is a cross-section taken through the distal end portion of the sheath of FIG. 3A;

FIG. 4 is a schematic view showing a first step of an example method for carrying out a cardiac procedure;

FIG. 5 is a schematic view showing a second step of the method of FIG. 4;

FIG. 6 is a schematic view showing a third step of the method of FIG. 4;

FIG. 7 is a schematic view showing a fourth step of the method of FIG. 4;

FIG. 8A is a perspective view of an imaging system displaying an en-face view of a patient's heart from a distal end portion of a sheath within a right atrium of the patient's heart;

FIG. 8B is a perspective view of an imaging system displaying a side view of an atrial septum of a patient's heart and a distal end portion of a sheath;

FIG. 8C is a perspective view of an imaging system displaying a rear view of a distal end portion of a sheath, and an atrial septum of a patient's heart;

FIG. 9 is a schematic view showing a fifth step of the method of FIG. 4;

FIG. 10 is a schematic view showing a sixth step of the method of FIG. 4; and

FIG. 11 is a schematic view showing a seventh step of the method of FIG. 4.

### DETAILED DESCRIPTION

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are medical sheaths (also referred to herein simply as “sheaths”) that can be used in medical procedures, such as cardiac procedures. For example, the sheaths can be used in transseptal perforation procedures, in which a sheath is advanced to the right atrium of a patient's heart via the femoral vein, and a perforation device (e.g. a radiofrequency (RF) perforation device) and dilator are guided through the sheath, to the right atrium. When the sheath is adjacent a target location in the right atrium, for example the fossa ovalis of the atrial septum, the perforation device can be advanced out of the sheath and used to create a perforation in the target location, and the dilator can be advanced out of the sheath to dilate the perforation. Such procedures can be carried out, for example, as a medical treatment, or to gain access to the left atrium for a subsequent medical treatment.

In general, the sheaths disclosed herein are configured to allow for non-fluoroscopic visualization of and/or determination of the location of the distal end portion of the sheath within the body. Furthermore, the sheaths disclosed herein are configured to allow for non-fluoroscopic visualization of the cardiac anatomy from viewpoints associated with the sheath (e.g. an en-face view of the fossa ovalis from the distal end portion of the sheath can be generated). More specifically, the sheaths disclosed herein can have a distal end portion that is at least partially echogenic (i.e. the distal end portion has at least a portion with an acoustic impedance that significantly differs from the acoustic impedance of blood or other tissue). When using a 3-dimensional ultrasound catheter (e.g. an intracardiac echocardiography (ICE) catheter or transesophageal echocardiography (TEE) catheter) to create a visual model of a patient's heart, the ultrasound catheter can also receive a reflected ultrasound signal from the distal end portion of the sheath. Based on the reflected ultrasound signal, a representation of the distal end portion of the sheath can be added to the visual model of the patient's heart (e.g. using standard image processing software features, such as edge detect, or feature detect). Optionally, the ultrasound system can utilize a known model of the sheath, in order to generate the representation of the distal end portion of the sheath based on the ultrasound signal received from the distal end of the sheath. For example, an image of the sheath can be added to the visual model of the patient's heart, and/or as mentioned above, a view of the patient's heart can be generated from a viewpoint associated with the sheath. This can enhance the safety of the procedure. For example, it can allow for a user (e.g. a physician) to ensure that or check whether the sheath is in the desired location with respect to the target location.

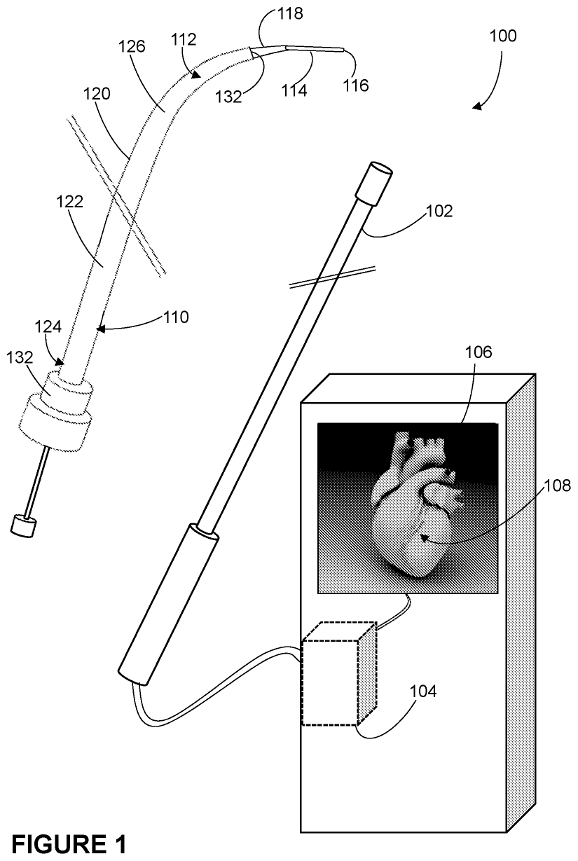

Referring now to FIG. 1, an example medical ultrasound system 100 (also referred to herein simply as a ‘system’) is shown. In the example shown, the system 100 includes a 3-dimensional ultrasound catheter 102 (also referred to herein as a U/S catheter), for emitting ultrasound signals to an anatomical volume and receiving reflected ultrasound signals from the anatomical volume. The U/S catheter 102 can be, for example, an ICE catheter or a TEE catheter. The system 100 further includes an ultrasound data processor 104 connected to the U/S catheter 102. Ultrasound data based on the reflected ultrasound signals is sent from the U/S catheter 102 to the ultrasound data processor 104, and the ultrasound data processor 104 receives and processes the ultrasound data using, for example, standard software features. The system 100 further includes an imaging system 106 connected to the ultrasound data processor 104 for generating a 3-dimensional visual model 108 of the anatomical volume, based on the processed data. Such U/S catheters 102, ultrasound data processors 104, and U/S imaging systems 106 are known in the art, are often sold together as an all-in-one system (e.g. such systems are sold by Siemens Healthcare GmbH or by General Electric Company), and will not be described in detail herein.

Referring still to FIG. 1, the system 100 further includes an elongate cylindrical hollow member, such as a sheath 110. In an alternative embodiment, the system may comprise an elongate member comprising a solid body (not shown). The sheath 110 has a distal end portion 112 that is at least partially echogenic (described in further detail below), for reflecting the ultrasound signals emitted by the U/S catheter 102. As described above, the U/S catheter 102 can receive the reflected signals, and the ultrasound data processor 104 can process ultrasound data received from the U/S catheter 102. Based on the processed data, the imaging system 106 can then generate a 3-dimensional visual model representing the positioning of the distal end portion 112 of the sheath 110 within the anatomical volume (as mentioned above, this can be done using standard image processing software features). For example, as will be described in further detail below, the imaging system 106 can generate an en-face view of the patient's heart from the distal end portion 112 of the sheath 110, generate a side view of the distal end portion 112 of the sheath 110 and an area of the patient's heart adjacent the distal end portion 112 of the sheath 110, and/or generate a rear-view of the distal end portion 112 of the sheath 110 and an area of the patient's heart adjacent the distal end portion 112 of the sheath 110.

The system can further include one or more treatment devices. Referring still to FIG. 1, in the example shown, the system 100 includes a radiofrequency perforation device 114 having a perforating tip 116, and a dilator 118, which can both be advanced towards the target location in the patient's heart via the sheath 110. The radiofrequency perforation device 114 can be connected to a radiofrequency generator (not shown), which can in turn be connected to one or more grounding pads (not shown). Radiofrequency perforation devices, generators, and grounding pads, as well as dilators, are known in the art, and will not be described in detail herein. Examples are sold by Baylis Medical Company, Inc. (Montreal, Canada), for example under the brand names NRG® Transseptal Platform, or SupraCross® Transseptal Platform.

In alternative examples, alternative or additional treatment devices may be part of the system.

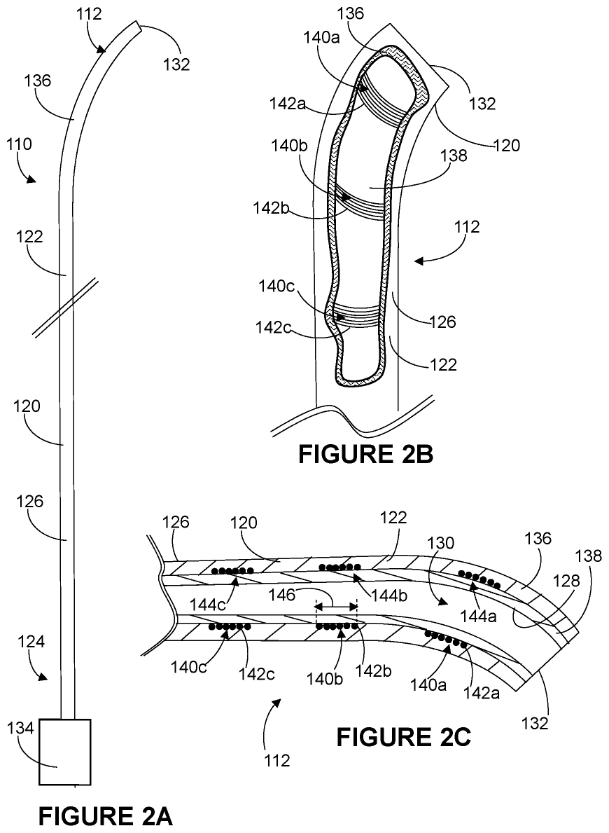

Referring now to FIGS. 2A to 2C, the sheath 110 is shown in greater detail. In the example shown, the sheath 110 includes an elongate member 120. The elongate member 120 has a sidewall 122 that extends longitudinally between a proximal end portion 124 of the elongate member 120 and the distal end portion 112 of the elongate member 120, and radially between an outer surface 126 of the elongate member 120 and an inner surface 128 (shown in FIG. 2C) of the elongate member 120. The inner surface 128 defines a lumen 130 (shown in FIG. 2C) of the elongate member 120. The lumen 130 extends through the elongate member 120 from the proximal end portion 124 to the distal end portion 112, and is open at the distal tip 132 of the sheath 110.

Referring to FIG. 2A, in the example shown, a handle 134 is mounted to the proximal end portion 124 of the elongate member 120. The handle 134 can include various hubs and/or ports and/or connection points (not shown) for connection to various external devices.

Referring still to FIGS. 2B and 2C, in the example shown, the elongate member 120 includes an outer tube 136, which defines the outer surface 126, and an inner liner 138 within the outer tube 136, which defines the inner surface 128. The inner liner 138 can be, for example, a polyimide or polytetrafluoroethylene liner, and the outer tube 136 can be, for example, made of a plastic such as high-density polyethylene (HDPE).

Referring to FIGS. 2B and 2C, in the example shown, the sheath 110 includes a set of echogenic bands associated with the distal end portion 112 and secured to the sidewall 122. In the example shown, the set of echogenic bands includes three echogenic bands—i.e. a first 140a, a second 140b, and a third 140c echogenic band—associated with the distal end portion 112. As used herein, the phrase “associated with” indicates that the echogenic bands 140a-140c are positioned to allow for the determination of the location of the distal end portion 112 using the ultrasound system 100, whether directly (e.g. as shown, where the echogenic bands 140a-140c are mounted directly to the distal end portion), or indirectly (e.g. in cases where echogenic bands are spaced from the distal end portion 112 and where a processing step, e.g. an extrapolation, is carried out to determine the location of the distal end portion 112 based on the location of the echogenic bands). In the example shown, the echogenic bands 140a-140c are mounted directly to the distal end portion 112 and are spaced apart along the distal end portion 112.

In alternative examples, the sheath can include another number of echogenic bands, e.g. one echogenic band, or two echogenic bands, or more than three echogenic bands. In examples in which less than three echogenic bands are used, a 3-dimensional representation of the sheath can be generated via the ultrasound system using known vectors associated with the distal end portion. For example, if the sheath includes only one echogenic band, a 3-dimensional representation of the sheath can be generated via the ultrasound system using two known vectors associated with the distal end portion of the sheath. Alternatively, or in addition, the ultrasound system can utilize a known model of the sheath, in order to generate the representation of the distal end portion of the sheath based on the ultrasound signal received from the distal end of the sheath.

The echogenic bands can be in various forms, such as a coil or a braid, and can extend around the entirety of the elongate member (e.g. around the outer surface), or can extend around only a portion of the elongate member (e.g. around only the inner liner, so that the echogenic bands are embedded in the sidewall). In an alternative embodiment, where the elongate member comprises a solid body, the echogenic band may be in the form of a disc, making up the entire cross-sectional area of the elongate member (not shown). Embedding the echogenic bands in the sidewall can allow for a smooth transition as the sheath is inserted through tissue. Referring still to FIGS. 2A to 2C, in the example shown, each echogenic band 140a, 140b, 140c is in the form of a respective coil 142a, 142b, 142c of echogenic material (e.g. a metal or alloy such as stainless steel or platinum-iridium), which extends around the inner liner 138 and is sandwiched between the inner liner 138 and outer tube 136, so that it is embedded in the sidewall 122. Embedding the echogenic bands in the sidewall allows for the puncturing device to be advanced through the inner lumen without being obstructed (i.e., snagged) by the echogenic bands. The echogenic bands 140a-140c are spaced apart, for example by up to 5 mm (e.g. between 1 mm and 3 mm, or about 2 mm), so that the first echogenic band 140a extends around a first portion 144a of the inner liner 138, the second echogenic band 140b extends around a second portion 144b of the inner liner 138, and the third echogenic band 140c extends around a third portion 144c of the inner liner 138. Each echogenic band 140a-140c can have a length 146 (where the length is measured longitudinally along the elongate member 120) of, for example, up to 5 mm (e.g. between 1 mm and 3 mm, or about 2 mm).

Optionally, in order to fabricate the sheath 110 of FIGS. 2A to 2C, the outer tube 136, coils 142a-142c of echogenic material, and inner liner 138 can first be assembled together. Then, the material of the outer tube 136 can be re-flowed (e.g. by the application of heat) to join the outer tube 136, coils 142a-142c of echogenic material, and inner liner 138.

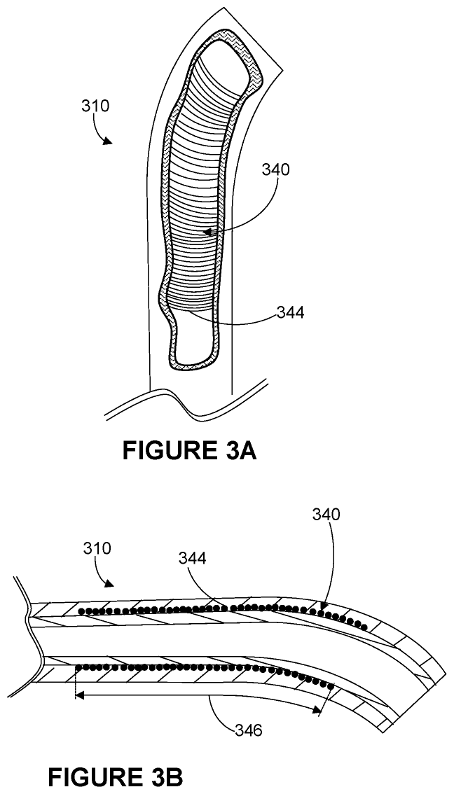

Referring now to FIGS. 3A and 3B, an alternative example of a sheath is shown. In FIGS. 3A and 3B, features that are like those of FIG. 2 will be referred to with like reference numerals, incremented by 100. The sheath 310 of FIGS. 3A and 3B includes a single band 340 of echogenic material, in the form of a coil 344 of echogenic material. The band 340 of echogenic material has a greater length 346 than the bands of echogenic material of FIG. 2.

In an alternative example (not shown), the bands of echogenic material can be formed by echogenic-filled polymer. For example, bands of tungsten-filled polymer can be incorporated into the sheath during manufacture, by assembling an outer tube, bands of tungsten-filled polymer, and an inner liner. Then, the material of the outer tube as well as the tungsten-filled polymer can be re-flowed (e.g. by the application of heat) to join the outer tube, bands of tungsten-filled polymer, and inner liner.

In another alternative example (not shown), instead of or in addition to embedding the echogenic bands in the sidewall, echogenic bands can be applied to the outer surface of the elongate member, and secured in place by gluing, welding, soldering, friction, and/or re-flowing. Optionally, the echogenic bands can be seated in a groove in the outer surface and can be swaged, so that the echogenic bands are flush with the outer surface.

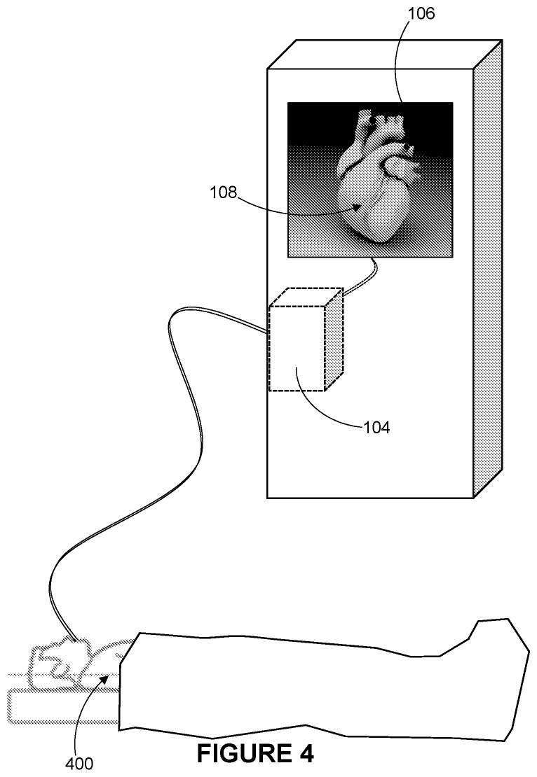

Referring now to FIGS. 4 to 11, a method for carrying out a cardiac procedure, specifically for creation of a transseptal perforation, will be described. As will be described in more detail, during the method, a representation of the distal end portion 112 of the sheath 110 can be added to the visual model 108 of the patient's heart. This can enhance the safety of the procedure. The method will be described with reference to the system 100 and sheath 110 as shown in FIGS. 1 to 2C; however, the method is not limited to being carried out with the system 100 and the sheath 110 of FIGS. 1 to 2C, and the system 100 and sheath 110 of FIGS. 1 to 2C are not limited to use according to the described method.

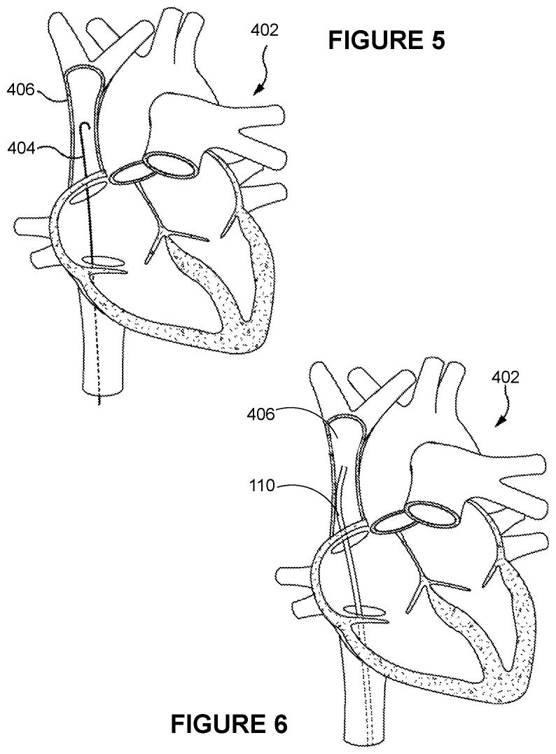

Referring to FIG. 4, as a first step, the U/S catheter 102 (not visible in FIG. 4) can be inserted into patient's body 400 and in conjunction with the ultrasound data processor 104 and imaging system 106, can be used to create the 3-dimensional visual model 108 of the patient's heart 402 (shown in FIG. 5). Optionally, the ultrasound data can be registered with computerized tomography (CT) and/or magnetic resonance imaging (MRI) data, to enhance the resolution of the visual model 108. Optionally, the visual model 108 can be used to check for a thrombus in the patient's heart 402 (this can be done repeatedly throughout the procedure).

Referring to FIG. 5, a guidewire 404 then be advanced via the femoral vein towards the heart 402, and “parked” in the superior vena cava (SVC) 406.

Referring to FIG. 6, with the dilator 118 (not visible in FIG. 6) shrouded in the sheath 110, the dilator 118 and sheath 110 can be advanced over the guidewire 404 towards the SVC 406. The guidewire 404 can then be removed, and the RF perforation device 114 (not visible in FIG. 6) can be advanced through the dilator 118 until the perforating tip 116 (not visible in FIG. 6) of the RF perforation device 114 is just shy of the distal end of the dilator 118.

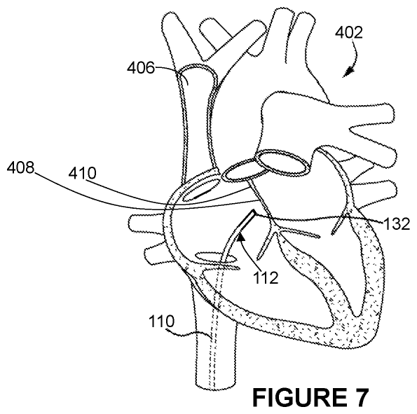

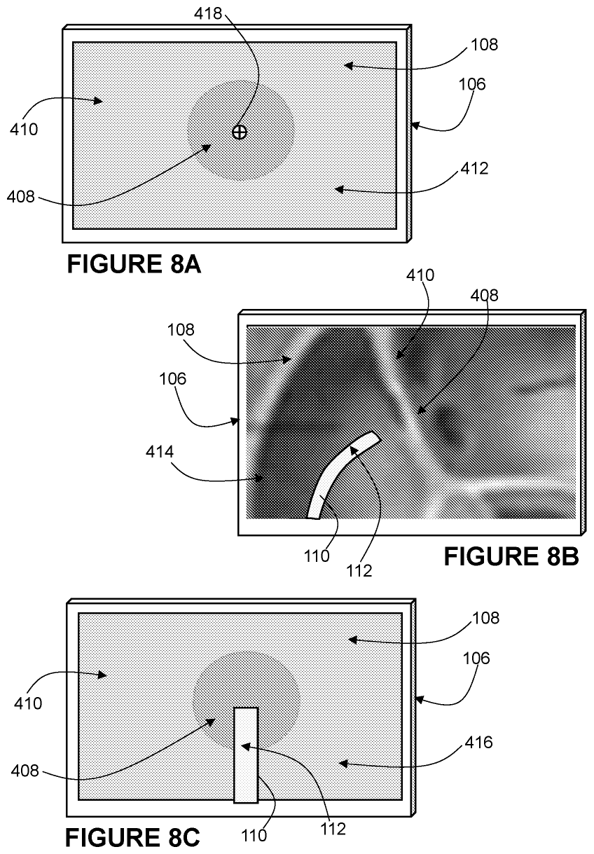

Referring now to FIG. 7, with the dilator 118 and radiofrequency perforation device 114 (not visible in FIG. 7) shrouded in the sheath 110, the distal end portion 112 of the sheath 110 can be advanced towards a target location in the patient's heart 402, to position the distal tip 132 of the sheath 110 adjacent the target location. The target location can be for example, the fossa ovalis 408 of the atrial septum 410. As the sheath 110 is advanced to the target location, the U/S catheter 102, ultrasound data processor 104, and imaging system 106 (not shown in FIG. 7) can be engaged, and used to determine a location of the distal end portion 112 of the sheath 110 with respect to the patient's heart 402. A representation of the distal end portion 112 can then be added to the visual model 108 of the patient's heart 402, as shown in FIGS. 8A to 8C. Optionally, the imaging system 106 can be updated in real-time as the sheath 112 is advanced towards the target location.

The representation of the distal end portion 112 of the sheath 110 can be added to the 3-dimensional visual model 108 of the patient's heart 402 in various ways. For example, as shown in FIG. 8A, the imaging system 106 can generate an en-face view 412 from the distal end portion 112 of the sheath 110 (not shown in FIG. 8A), to show the atrial septum 410 and fossa ovalis 408. For further example, as shown in FIG. 8B, the imaging system 106 can generate a side view 414 of the distal end portion 112 of the sheath 110 and an area of the patient's heart adjacent the distal end portion (e.g. the atrial septum 410 and fossa ovalis 408). For further example, as shown in FIG. 8C, the imaging system 106 can generate a rear-view 416 of the distal end portion 112 of the sheath 110 and an area of the patient's heart adjacent the distal end portion (e.g. the atrial septum 410 and fossa ovalis 408). Optionally, the various views can be presented simultaneously, for example in a split-screen view. Optionally, the user can select a desired view from multiple options. Optionally, the view can be fixed on the target location while the sheath 110 is advanced towards the target location.

In some examples, the system 100 can be configured to add to the visual model 108 a representation of a predicted contact location of the treatment device (in this case the perforation device 114) and the target location. For example, as shown in FIG. 8A, prior to the perforation device 114 being advanced out of the sheath 110, the imaging system 106 can add a mark 418 to the 3-dimensional visual model 108, indicating the location where the perforation device 114 is predicted to contact tissue when it is advanced. This prediction can be made based the location of the sheath 110 as determined by the system 100, and based on known parameters (e.g. curvature) of the perforation device 114.

If the mark 418 is in the desired position with respect to the target location, e.g. on the fossa ovalis 408 as shown, the perforation device 114 can be advanced out of the sheath 110. Alternatively, if the mark 418 is not in the desired position, e.g. spaced from the fossa ovalis 408, then the position of the sheath 110 can be adjusted until the mark 418 is in the desired position, and the perforation device 114 can then be advanced out of the sheath 110.

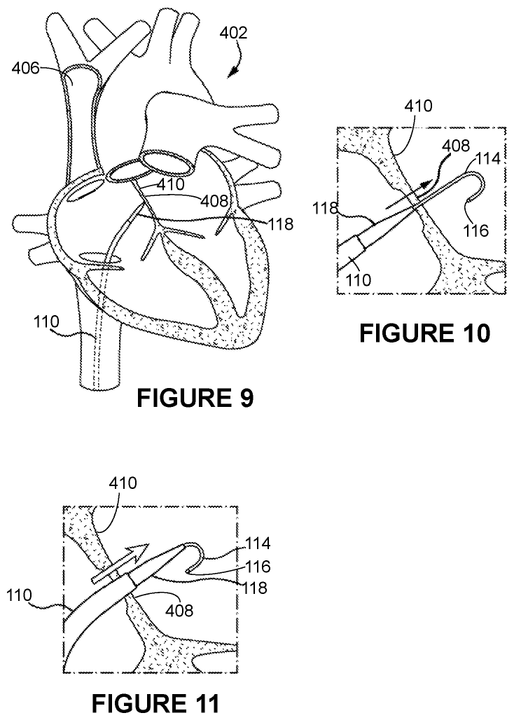

Referring to FIGS. 9 to 11, the perforation device 114 can then be engaged to perforate the fossa ovalis 408, and the dilator 118 can be advanced from the sheath 110 to dilate the perforation. The sheath 110 can then be advanced through the perforation, to the left atrium.

Once access to the left atrium has been gained, a subsequent medical treatment (not shown) can be carried out. Optionally, the echogenicity of the distal end portion 110 of the sheath 110 can continue to be used during the subsequent medical treatment. For example, the subsequent medical treatment can be a pulmonary vein isolation (PVI) procedure using a balloon-based system. In such cases, another en-face view of the heart from the distal end portion 112 of the sheath 110 can be generated, in order to allow a user to visualize whether blood is leaking past the balloon (which may impact lesion quality).

Upon completion of the medical treatment or at a desired time, the dilator 118, perforation device 114, and sheath 110 can be withdrawn from the heart 402. Optionally, during withdrawal, the representation of the distal end portion 112 of the sheath 110 can continue to be added to the visual model 108 of the patient's heart 402.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A medical sheath, comprising:
an elongate member having a sidewall extending longitudinally between a proximal end portion of the elongate member and an opposed distal end portion of the elongate member, and radially between an outer surface of the elongate member and an inner surface of the elongate member;
a lumen defined by the inner surface and extending through the elongate member from the proximal end portion to the distal end portion; and
at least a first echogenic band associated with the distal end portion and secured to the sidewall.

2. The medical sheath of claim 1, wherein the first echogenic band comprises a first coil of echogenic material extending around at least a first portion of the elongate member.

3. The medical sheath of any one of claims 1 to 2, further comprising a second echogenic band associated with the distal end portion and spaced from the first echogenic band.

4. The medical sheath of claim 3, wherein the second echogenic band comprises a second coil of echogenic material extending around at least a second portion of the elongate member.

5. The medical sheath of any one of claims 1 to 3, further comprising a third echogenic band associated with the distal end portion and spaced from the first echogenic band and the second echogenic band.

6. The medical sheath of claim 5, wherein the third echogenic band comprises a third coil of echogenic material extending around at least a third portion of the elongate member.

7. The medical sheath of any one of claims 1 to 6, wherein said echogenic band comprises a braid of echogenic material extending around at least a first portion of the elongate member.

8. The medical sheath of any one of claims 1 to 7, wherein said echogenic band is embedded in the sidewall.

9. The medical sheath of claim 8, wherein the elongate member comprises an inner liner and an outer tube, and said echogenic band is positioned between the inner liner and outer tube.

10. The medical sheath of any one of claims 1 to 7, wherein said echogenic band is positioned on the outer surface of the elongate member.

11. The medical sheath of any one of claims 1 to 10, wherein said echogenic band comprises a band of echogenic-filled polymer.

12. A medical ultrasound system, comprising:
a 3-dimensional ultrasound catheter for emitting ultrasound signals to an anatomical volume and receiving reflected ultrasound signals from the anatomical volume;
a medical sheath having a distal end portion that is at least partially echogenic for reflecting the ultrasound signals emitted by the 3-dimensional ultrasound catheter;
an ultrasound data processor for receiving ultrasound data from the 3-dimensional ultrasound catheter and processing the ultrasound data, wherein the ultrasound data is based on the reflected ultrasound signals; and
an imaging system connected to the ultrasound data processor for generating a 3-dimensional visual model representing the positioning of the distal end portion of the medical sheath within the anatomical volume, based on the processed data.

12. A medical ultrasound system, comprising:
a 3-dimensional ultrasound catheter for emitting ultrasound signals to an anatomical volume and receiving reflected ultrasound signals from the anatomical volume;
a medical sheath having a distal end portion that is at least partially echogenic for reflecting the ultrasound signals emitted by the 3-dimensional ultrasound catheter;
an ultrasound data processor for receiving ultrasound data from the 3-dimensional ultrasound catheter and processing the ultrasound data, wherein the ultrasound data is based on the reflected ultrasound signals; and
an imaging system connected to the ultrasound data processor for generating a 3-dimensional visual model representing the positioning of the distal end portion of the medical sheath within the anatomical volume, based on the processed data.

13. The medical ultrasound system of claim 12, wherein the medical sheath comprises:
an elongate member having a sidewall extending longitudinally between a proximal end portion of the elongate member and an opposed distal end portion of the elongate member, and radially between an outer surface of the elongate member and an inner surface of the elongate member;
a lumen defined by the inner surface and extending through the elongate member from the proximal end portion to the distal end portion; and
at least a first echogenic band associated with the distal end portion and secured to the sidewall.

14. The medical ultrasound system of claim 13, wherein the first echogenic band comprises a first coil of echogenic material extending around at least a first portion of the elongate member.

15. The medical ultrasound system of any one of claims 13 to 14, further comprising a second echogenic band associated with the distal end portion and spaced from the first echogenic band.

16. The medical ultrasound system of claim 15, wherein the second echogenic band comprises a second coil of echogenic material extending around at least a second portion of the elongate member.

17. The medical sheath of any one of claims 13 to 16, further comprising a third echogenic band associated with the distal end portion and spaced from the first echogenic band and the second echogenic band.

18. The medical sheath of claim 17, wherein the third echogenic band comprises a third coil of echogenic material extending around at least a third portion of the elongate member.

19. The medical sheath of any one of claims 13 to 18, wherein said echogenic band comprises a braid of echogenic material extending around at least a first portion of the elongate member.

20. The medical sheath of any one of claims 13 to 19, wherein said echogenic band is embedded in the sidewall.

21. The medical sheath of claim 20, wherein the elongate member comprises an inner liner and an outer tube, and said echogenic band is positioned between the inner liner and outer tube.

22. The medical sheath of any one of claims 13 to 19, wherein said echogenic band is positioned on the outer surface of the elongate member.

23. The medical sheath of any one of claims 13 to 22, wherein said echogenic band comprises a band of echogenic-filled polymer.

24. A method for carrying out a cardiac procedure, comprising:
a. inserting a 3-dimensional ultrasound catheter into patient's body and using the 3-dimensional ultrasound catheter to create a visual model of the patient's heart;
b. inserting a medical sheath into the patient's heart and advancing a distal end portion of the medical sheath towards a target location in the patient's heart, wherein the distal end portion is at least partially echogenic;
c. using the 3-dimensional ultrasound catheter to determine a location of the distal end portion of the medical sheath with respect to the patient's heart; and
d. adding a representation of the distal end portion to the visual model of the patient's heart.

25. The method of claim 24, wherein step d. comprises generating an en-face view of the patient's heart from the distal end portion.

26. The method of claim 24, wherein step d. comprises generating a side view of the distal end portion and an area of the patient's heart adjacent the distal end portion.

27. The method of claim 24, wherein step d. comprises generating a rear-view of the distal end portion and an area of the patient's heart adjacent the distal end portion.

28. The method of claim 24, wherein the method further comprises e. advancing a treatment device out of the distal end portion towards the target location, and f. using the treatment device to treat the target location.

29. The method of claim 28, wherein prior to step e., the method comprises adding to the visual model a representation of a predicted contact location of the treatment device and the patient's heart.

30. The method of claim 28, wherein the treatment device is a radiofrequency perforation device and the target location is a fossa ovalis.

31. The method of claim 28, wherein the treatment device is a balloon-based system and the target location is a pulmonary vein.

32. A kit of parts for a carrying out a cardiac procedure, comprising:
a medical sheath, the medical sheath comprising: an elongate member having a sidewall extending longitudinally between a proximal end portion of the elongate member and an opposed distal end portion of the elongate member, and radially between an outer surface of the elongate member and an inner surface of the elongate member; a lumen defined by the inner surface and extending through the elongate member from the proximal end portion to the distal end portion; and at least a first echogenic band associated with the distal end portion and secured to the sidewall;
a radiofrequency perforation device having a perforating tip, the radiofrequency perforation device receivable in the lumen with the perforating tip at the distal end portion.

33. The kit of parts of claim 32, wherein the first echogenic band comprises a first coil of echogenic material extending around at least a first portion of the elongate member.

34. The kit of parts of any one of claims 32 to 33, further comprising a second echogenic band associated with the distal end portion and spaced from the first echogenic band.

35. The kit of parts of claim 34, wherein the second echogenic band comprises a second coil of echogenic material extending around at least a second portion of the elongate member.

36. The kit of parts of any one of claims 32 to 35, further comprising a third echogenic band associated with the distal end portion and spaced from the first echogenic band and the second echogenic band.

37. The kit of parts of claim 36, wherein the third echogenic band comprises a third coil of echogenic material extending around at least a third portion of the elongate member.

38. The kit of parts of any one of claims 32 to 37, wherein said echogenic band comprises a braid of echogenic material extending around at least a first portion of the elongate member.

39. The kit of parts of any one of claims 32 to 38, wherein said echogenic band is embedded in the sidewall.

40. The kit of parts of claim 39, wherein the elongate member comprises an inner liner and an outer tube, and said echogenic band is positioned between the inner liner and outer tube.

41. The kit of parts of any one of claims 32 to 38, wherein said echogenic band is positioned on the outer surface of the elongate member.

42. The kit of parts of any one of claims 32 to 41, wherein said echogenic band comprises a band of echogenic-filled polymer.

43. An echogenic device, comprising:
an elongate member having a solid body extending longitudinally between a proximal end portion of the elongate member and an opposed distal end portion of the elongate member; and
at least a first echogenic band associated with the distal end portion and secured to the solid body of the elongate member.

44. The echogenic device of claim 43, wherein the first echogenic band comprises a first coil of echogenic material extending around at least a first portion of the elongate member.

45. The echogenic device of any one of claims 43 to 44, further comprising a second echogenic band associated with the distal end portion and spaced from the first echogenic band.

46. The echogenic device of claim 45, wherein the second echogenic band comprises a second coil of echogenic material extending around at least a second portion of the elongate member.

47. The echogenic device of any one of claims 43 to 45, further comprising a third echogenic band associated with the distal end portion and spaced from the first echogenic band and the second echogenic band.

48. The echogenic device of claim 47, wherein the third echogenic band comprises a third coil of echogenic material extending around at least a third portion of the elongate member.

49. The echogenic device of any one of claims 43 to 48, wherein said echogenic band comprises a braid of echogenic material extending around at least a first portion of the elongate member.

50. The echogenic device of any one of claims 43 to 49, wherein said echogenic band is embedded in the solid body.

51. The echogenic device of any one of claims 43 to 49, wherein said echogenic band is positioned on an outer surface of the elongate member.

52. The echogenic device of any one of claims 43 to 50, wherein said echogenic band comprises a solid disc making up a cross-sectional area of the elongate member.

53. The echogenic device of claims 43 to 49, wherein the catheter further comprises a lumen extending through the elongate member from the proximal end portion to the distal end portion.

54. The echogenic device of claim 53, wherein the elongate member comprises an inner liner and an outer tube, and said echogenic band is positioned between the inner liner and outer tube.

55. The echogenic device of any one of claims 43 to 54, wherein said echogenic band comprises a band of echogenic-filled polymer.

57. A medical ultrasound system, comprising:
a 3-dimensional ultrasound catheter for emitting ultrasound signals to an anatomical volume and receiving reflected ultrasound signals from the anatomical volume;
an echogenic device having a distal end portion that is at least partially echogenic for reflecting the ultrasound signals emitted by the 3-dimensional ultrasound catheter;
an ultrasound data processor for receiving ultrasound data from the 3-dimensional ultrasound catheter and processing the ultrasound data, wherein the ultrasound data is based on the reflected ultrasound signals; and
an imaging system connected to the ultrasound data processor for generating a 3-dimensional visual model representing the positioning of the distal end portion of the medical sheath within the anatomical volume, based on the processed data.

58. The medical ultrasound system of claim 57, wherein the echogenic device comprises:
an elongate member having a solid body extending longitudinally between a proximal end portion of the elongate member and an opposed distal end portion of the elongate member; and
at least a first echogenic band associated with the distal end portion and secured to the solid body.

59. The medical ultrasound system of any one of claim 58, wherein the first echogenic band comprises a first coil of echogenic material extending around at least a first portion of the elongate member.

60. The medical ultrasound system of any one of claims 58 to 59, further comprising a second echogenic band associated with the distal end portion and spaced from the first echogenic band.

61. The medical ultrasound system of claim 60, wherein the second echogenic band comprises a second coil of echogenic material extending around at least a second portion of the elongate member.

62. The echogenic device of any one of claims 58 to 61, further comprising a third echogenic band associated with the distal end portion and spaced from the first echogenic band and the second echogenic band.

63. The echogenic device of claim 62, wherein the third echogenic band comprises a third coil of echogenic material extending around at least a third portion of the elongate member.

64. The echogenic device of any one of claims 58 to 563, wherein said echogenic band comprises a braid of echogenic material extending around at least a first portion of the elongate member.

65. The echogenic device any one of claims 59 to 64, wherein said echogenic band is embedded in the solid body.

66. The echogenic device in any one of claims 59 to 64, wherein said echogenic band is positioned on an outer surface of the elongate member.

67. The echogenic device of any one of claims 58 to 66, wherein said echogenic band comprises a band of echogenic-filled polymer.

68. The medical ultrasound system of any one of claims 58 to 66, wherein the echogenic device further comprises a lumen extending through the elongate member from the proximal end portion to the distal end portion.

69. The echogenic device of claim 68, wherein the elongate member comprises an inner liner and an outer tube, and said echogenic band is positioned between the inner liner and outer tube.

70. The medical ultrasound system of claim 58, wherein said echogenic band comprises a solid disc making up a cross-sectional area of the elongate member.

71. A method for carrying out a cardiac procedure, comprising:
a. inserting a 3-dimensional ultrasound catheter into patient's body and using the 3-dimensional ultrasound catheter to create a visual model of the patient's heart;
b. inserting an echogenic device into the patient's heart and advancing a distal end portion of the echogenic device towards a target location in the patient's heart, wherein the distal end portion is at least partially echogenic;
c. using the 3-dimensional ultrasound catheter to determine a location of the distal end portion of the echogenic device with respect to the patient's heart; and
d. adding a representation of the distal end portion to the visual model of the patient's heart.

72. The method of claim 71, wherein step d. comprises generating an en-face view of the patient's heart from the distal end portion.

73. The method of claim 71, wherein step d. comprises generating a side view of the distal end portion and an area of the patient's heart adjacent the distal end portion.

74. The method of claim 71, wherein step d. comprises generating a rear-view of the distal end portion and an area of the patient's heart adjacent the distal end portion.

75. The method of claim 71, wherein the method further comprises e. advancing a treatment device out of the distal end portion towards the target location, and f. using the treatment device to treat the target location.

76. The method of claim 75, wherein prior to step e., the method comprises adding to the visual model a representation of a predicted contact location of the treatment device and the patient's heart.

77. The method of claim 75 wherein the treatment device is a radiofrequency perforation device and the target location is a fossa ovalis.

78. The method of claim 75, wherein the treatment device is a balloon-based system and the target location is a pulmonary vein.

80. A kit of parts for a carrying out a cardiac procedure, comprising:
an echogenic device, the echogenic device comprising: an elongate member having a solid body extending longitudinally between a proximal end portion of the elongate member and an opposed distal end portion of the elongate member; and at least a first echogenic band associated with the distal end portion; and,
a radiofrequency perforation device having a perforating tip.

81. The kit of parts of claim 80, wherein the first echogenic band comprises a first coil of echogenic material extending around at least a first portion of the elongate member.

82. The kit of parts of any one of claims 80 to 81, further comprising a second echogenic band associated with the distal end portion and spaced from the first echogenic band.

83. The kit of parts of claim 82, wherein the second echogenic band comprises a second coil of echogenic material extending around at least a second portion of the elongate member.

84. The kit of parts of any one of claims 80 to 82, further comprising a third echogenic band associated with the distal end portion and spaced from the first echogenic band and the second echogenic band.

85. The kit of parts of claim 84, wherein the third echogenic band comprises a third coil of echogenic material extending around at least a third portion of the elongate member.

86. The kit of parts of any one of claims 80 to 85, wherein said echogenic band comprises a braid of echogenic material extending around at least a first portion of the elongate member.

87. The kit of parts of any one of claims 80 to 86, wherein said echogenic band is embedded in the solid body.

88. The kit of parts of any one of claim 80, wherein said echogenic band comprises a band of echogenic-filled polymer.

89. The kit of parts of any one of claims 80 to 86, wherein said echogenic band is positioned on an outer surface of the elongate member.

90. The kit of parts of claims 80 to 88, wherein the echogenic device comprises a lumen defined by the inner surface and extending through the elongate member from the proximal end portion to the distal end portion.

91. The kit of parts of claim 90, wherein the elongate member comprises an inner liner and an outer tube, and said echogenic band is positioned between the inner liner and outer tube.

92. The kid of parts of claim 90, wherein the radiofrequency perforation device receivable in the lumen with the perforating tip at the distal end portion.

93. The kit of parts of claim 80, wherein said echogenic band comprises a solid disc making up a cross-sectional area of the elongate member.
